(19) United States
(12) Patent Application Publication
(10) Pub. No.: US 2022/0142626 A1
(43) Pub. Date: May 12, 2022

(54) SURGICAL INSTRUMENTS FOR GRASPING TISSUE AND SENSING TISSUE PROPERTIES

(71) Applicant: Covidien LP, Mansfield, MA (US)

(72) Inventors: Saumya Banerjee, Collinsville, CT (US); Jacob C. Baril, Norwalk, CT (US); Justin J. Thomas, New Haven, CT (US); Garrett P. Ebersole, Hamden, CT (US); Roy J. Pilletere, Middletown, CT (US); Matthew A. Dinino, Newington, CT (US); Nicolette LaPierre Roy, Windsor Locks, CT (US)

(21) Appl. No.: 17/515,723

(22) Filed: Nov. 1, 2021

Related U.S. Application Data
(60) Provisional application No. 63/111,715, filed on Nov. 10, 2020.

(51) Int. Cl. A61B 17/00

(52) U.S. Cl. CPC A61B 17/00234; A61B 2017/00867; A61B 2017/00327; A61B 2017/00022; A61B 2017/00314; A61B 2017/00296

(57) ABSTRACT

An endoscopic surgical instrument includes an elongated shaft and a pair of elongated arms extending through the shaft. The arms have a distal end portion configured to grasp tissue therebetween. The distal end portion of at least one of the arms is equipped with a sensor for sensing a property of the tissue.

### CROSS-REFERENCE TO RELATED APPLICATIONS

This application claims the benefit of and priority to U.S. Provisional Patent Application, filed Nov. 10, 2020, the entire contents of which is incorporated by reference herein.

### FIELD

The present technology is generally related to surgical instruments for determining and monitoring characteristics of tissue in preparation for performing various surgical procedures.

### BACKGROUND

Some surgical procedures, such as, for example, colorectal surgery, requires anastomosis, which involves resecting a piece of diseased tissue and creating a new connection between two presumably healthy segments. Typically, before performing the anastomosis, the amount of tissue to be resected is estimated using visual indicia of the tissue. The goal is to preserve as much healthy tissue as possible while at the same time removing all of the diseased tissue.

A risk involved in performing an anastomotic procedure is anastomotic leaks typically caused by a failure to resect all of the diseased tissue. Current methods used in estimating the amount of tissue to be resected during an anastomotic procedure are sometimes inadequate in preventing all anastomotic leaks. Additionally, the health and viability of tissue sections may be compromised by excessive tension or insufficient blood flow in the newly attached sections.

### SUMMARY

The techniques of this disclosure generally relate to minimally invasive surgical instruments for grasping tissue and sensing properties of the tissue to assist in performing a surgical procedure.

According to one aspect of the disclosure, a surgical instrument is provided and includes an elongated shaft defining a passageway therethrough and a pair of first and second elongated arms extending through the shaft. Each of the first and second arms has a generally arcuate, distal end portion configured to extend distally out of the shaft and surround tissue. The distal end portion of the first arm has a sensor configured to sense a property of the tissue. Each of the first and second arms has a distal tip fabricated from a magnetic material such that the distal tips are magnetically attracted to one another.

In aspects, the distal end portion of the second arm may have an emitter configured to emit a signal sensed by the sensor.

In aspects, the sensor may be a photodetector, and the emitter may be an infrared emitter or an LED emitter.

In aspects, the surgical instrument may further include a processor in communication with the sensor and the emitter. The processor is configured to determine the property of the tissue based on information received from the sensor.

In aspects, the processor may be configured to cause the emitter to emit the signal upon the distal tips engaging one another.

In aspects, the distal tip of the first arm and the distal tip of the second arm may be oriented toward one another.

In aspects, the first and second arms may be configured to translate relative to the shaft between a retracted position and an extended position. In the retracted position, the distal end portion of each of the first and second arms are received within the shaft, and in the extended position, the distal end portion of each of the first and second arms extends distally out of the shaft.

In aspects, the distal end portion of the first arm may have a linear segment fabricated from a rigid material, and a remainder of the distal end portion of the first arm may be arcuate and fabricated from a flexible material.

In aspects, the linear segment may house the sensor.

In aspects, the distal end portion of the second arm may be fabricated from the flexible material.

In aspects, the flexible material may be a shape memory material such that the distal end portion of the first and second arms are biased toward a generally arcuate shape.

In aspects, the tissue property may be blood perfusion, oxygen concentration, and/or oxygen pressure.

In accordance with another aspect of the disclosure, a tissue grasper for sensing tissue properties is provided and includes a handle portion having a processor, an elongated shaft extending distally from the handle portion, and a pair of first and second elongated arms extending through the shaft. Each of the first and second arms has a distal end portion configured to extend distally out of the shaft and to surround tissue. The distal end portion of the first arm has a sensor in communication with the processor. The sensor is configured to sense a property of the tissue, and the distal end portion of the second arm has an emitter configured to emit a signal sensed by the sensor.

In aspects, the first arm may have a magnetic distal tip, and the second arm may have a magnetic distal tip such that the distal tips are magnetically attracted to one another.

In aspects, the distal tips may be arcuate and oriented toward one another.

In aspects, the sensor may be a plurality of photodetectors, and the emitter may be a plurality of infrared or LED emitters.

In aspects, the processor may be in communication with the plurality of emitters and configured to determine the property of the tissue based on information received from the plurality of photodetectors.

In aspects, the processor may be configured to cause the plurality of emitters to emit the signal upon the distal end portions of the first and second arms engaging one another.

In aspects, the first and second arms may be configured to translate relative to the shaft between a retracted position, in which the distal end portion of each of the first and second arms is received within the shaft, and an extended position, in which the distal end portion of each of the first and second arms extends distally out of the shaft.

In aspects, the distal end portion of the first arm may be a linear segment fabricated from a rigid material, and a remainder of the distal end portion of the first arm may be arcuate and fabricated from a flexible material.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Embodiments of the disclosure are described herein with reference to the accompanying drawings, wherein:

FIG. 1 is a side, perspective illustrating a distal end portion of an exemplary surgical instrument for grasping tissue and sensing tissue properties;

FIG. 2 is a perspective view illustrating the distal end portion of the surgical instrument of FIG. 1 grasping tissue;

FIG. 3 is a front view illustrating an exemplary surgical bracelet, in an opened state, for wrapping about tissue and sensing tissue properties;

FIG. 4 is a top view illustrating the surgical bracelet of FIG. 3;

FIG. 5 is a side view of an end of the surgical bracelet of FIG. 3;

FIG. 6 is a perspective view illustrating the surgical bracelet of FIG. 3 disposed about an esophagus; and

FIG. 7 is a top view illustrating the surgical bracelet of FIG. 3 wrapped about an anastomosis.

### DETAILED DESCRIPTION

Embodiments of the presently disclosed surgical instruments and methods of treatment are described in detail with reference to the drawings, in which like reference numerals designate identical or corresponding elements in each of the several views. As used herein, the term “distal” refers to that portion of a structure that is closer to a surgical site, while the term “proximal” refers to that portion of a structure that is further from the surgical site. The term “clinician” refers to a doctor, nurse, or other care provider and may include support personnel.

Clinicians rely on qualitative tools such as an indocyanine green, radio-sensitive dye along with visual cues of tissue color and presence of blood (e.g., oozing) to determine if blood flow is adequate in the desired region. Sometimes, the only way to verify a repair is to test it post intervention using leak tests or immersion tests to check for bubbles. However, this option is not always feasible. There are several factors that determine whether an anastomosis or intervention is going to heal and not result in a leak. Some factors depend on patient criteria such as obesity, high blood pressure, smoking history, and/or the presence of other comorbidities. From the clinician's perspective, at the site of the intervention, the mechanical tension on the tissue as well as oxygen concentration/blood flow may also have a significant impact on the tissue healing process.

The present technology described herein provides a pair of semi-rigid endoscopic tubes contained within an about a 12 m to about a 15 mm diameter shaft or cannula. A first of the flexible tubes or arms supports LED emitters and a second of the flexible tubes or arms supports a photodetector array to capture the signal emitted by the LED emitters. Each of the arms has an arcuate distal tip configured to engage one another while clamping about tissue requiring circumferential evaluation. The distal tips may be magnetic to assist in closing and maintaining the distal tips engaged to one another about the tissue. In aspects, the arms may be equipped with any other suitable low-power sensors and may be embedded within the arms. The sensors may have flexible circuits to allow for flexing of the arms. It is contemplated that the magnetic distal tips completes a power circuit to allow sensor use upon the magnetic distal tips engaging one another.

The surgical instrument allows for monitoring tissues/organs that may be hidden from the visual field for signs of early necrosis or reduced perfusion. The surgical instrument may be introduced into the surgical site via a laparoscopic port and may be adjusted intraoperatively using exist laparoscopic tools such as a grasper. By providing tissue parameters of the surgical site, the surgical instrument gives a clinician the information needed to adjust or proceed with the intervention. This may be useful for determining the success of various surgical procedures, such as esophagectomies, colon resections, enterotomies, etc., which depend on uncompromised blood perfusion to improve clinical outcomes. The surgical instrument may also be incorporated into a surgical robotic system to assist with robotic surgical procedures.

Also provided herein is an expandable bracelet equipped with an array of sensors, such as, for example, IR or near-IR photodiodes and detectors configured to interrogate tissue or organs of interest. The bracelet may be flexible to be used on varying sizes and challenging tissues or organs that require circumferential evaluation. The bracelet allows for monitoring tissues/organs that are hidden from the visual field for signs of early necrosis or reduced perfusion. The bracelet may comprise a plurality of discrete units detachably coupled to one another so that the circumference of the bracelet may be adjusted by adding or removing the discrete units. The bracelet may be introduced via a laparoscopic port and assembled using any suitable laparoscopic tool or grasper in open procedures installed using the clinician's hands or a robotic instrument.

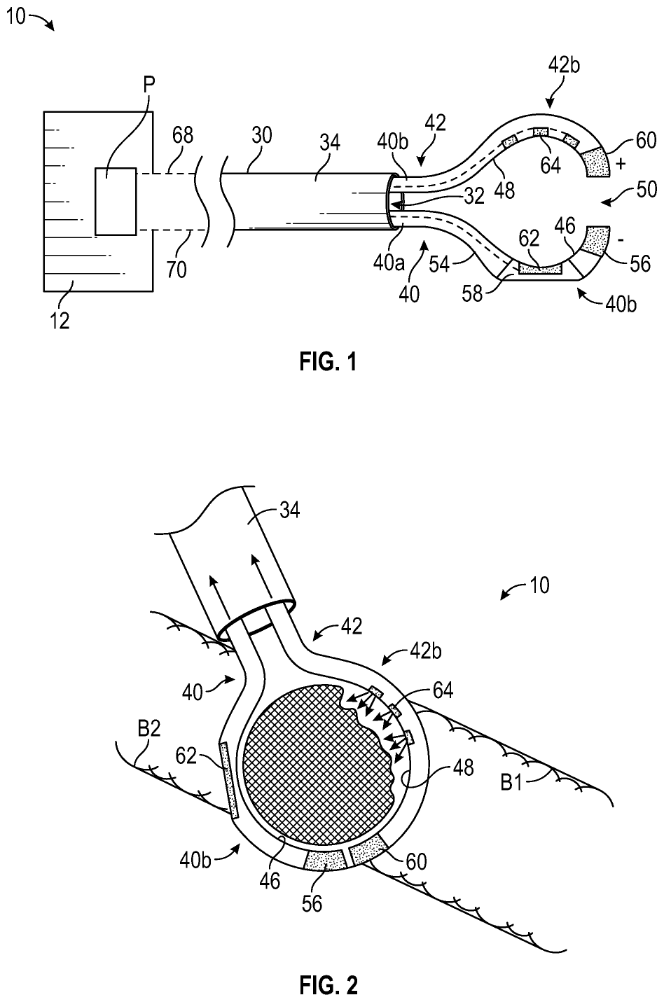

FIGS. 1 and 2 illustrate a surgical instrument, such as, for example, a tissue grasper 10 configured to grasp tissue and sense a multiplicity of biological parameters of the grasped tissue to assist a surgeon in performing a surgical procedure, for example, an anastomotic surgical procedure, as will be described in detail herein. The tissue grasper 10 generally includes a handle portion 12, an elongated shaft 30, and a pair of opposed first and second elongate arms 40, 42 received within the shaft 30. In aspects, the surgical instrument 10 may be devoid of a handle portion and instead may be coupled to and operated by a surgical robotic system. In some aspects, tissue grasper 10 may include a display (not shown) or may be configured to be connected to or be in communication with a tablet, a cell phone, a computer monitor, a laptop, or any suitable display device. Tissue grasper 10 may be connected to any of the aforementioned display devices via USB wires, Wi-Fi, or the like.

The elongated shaft 30 of tissue grasper 10 extends distally from the handle portion 12. The shaft 30 may have a tubular configuration and defines a longitudinally-extending passageway 32. The shaft 30 may have a diameter from about 12 mm to about 15 mm to allow for a laparoscopic application of the tissue grasper 10. In aspects, the diameter or overall profile of the shaft 30 may be more than 15 mm or less than 12 mm.

The arms 40, 42 of the tissue grasper 10 each extend distally from the handle portion 12, through the shaft 30, and terminate in a distal end portion 40b, 42b that is configured to selectively extend distally out of a distal end portion 34 of the shaft 30. The arms 40, 42 each have a proximal end portion 40a, 42a operably coupled to a movable handle or trigger (not explicitly shown) of the handle portion 12 configured to proximally or distally translate the arms 40, 42 relative to the shaft 30 to move the distal end portion 40b, 42b of the arms 40, 42 from a retracted position within the passageway 32 of the shaft 30 to an extended position outside of the shaft 30.

The distal end portion 40b, 42b of each of the arms 40, 42 oppose one another and may have a generally arcuate shape. The distal end portion 40b, 42b of each of the arms 40, 42 has a generally concave, tissue-contacting surface 46, 48, respectively, oriented toward one another such that the arms 40, 42 cooperatively define a circular opening 50 configured for receipt of a tissue segment, such as a bowel segment, an esophagus segment, or the like. The distal end portion 40b, 42b of each of the arms 40, 42 are flexible and resiliently biased toward an expanded state (FIGS. 1 and 2) while being configured to transition to a collapsed state (not explicitly shown) when the distal end portions 40b, 42b are retracted into the shaft 30. In aspects, the distal end portion 40b, 42b of the first and second arms 40, 42 may be partially fabricated from a shape memory material (e.g., nickel-titanium) that biases the distal end portions 40b, 42b toward the arcuate, expanded state. In aspects, the distal end portion 40b, 42b of the first and second arms 40, 42 may be fabricated from other suitable materials, such as other metals or plastics.

The distal end portion 40b of the first arm 40 includes a proximal segment 54, a distal tip 56, and an intermediate segment 58 disposed between the proximal segment 54 and the distal tip 56. The proximal segment 54 extends distally from the proximal end portion 40a of the first arm 40 and curves outwardly, the intermediate segment 58 extends distally from the proximal segment 54 and is linear or substantially linear, and the distal tip 56 extends distally from the intermediate segment 58 and curves inwardly therefrom. As such, the distal end portion 40b of the first arm 40 has a substantially or generally arcuate shape along its length. The proximal segment 54 and the distal tip 56 are each fabricated from a flexible material (e.g., the shape memory material) and the intermediate segment 58 may be fabricated from a relatively rigid material (e.g., steel) such that intermediate segment 58 maintains its linear shape during use.

It is contemplated that the distal end portion 42b of the second arm 42 may be constructed in the same or similar manner as the distal end portion 40b of the first arm 40b. In aspects, the entire distal end portion 40b, 42b of each of the first and second arms 40, 42 may be fabricated from the same material, such as, for example, a flexible material. In aspects the arms 40, 42 may be tubes, rods, or the like.

The distal end portion 42b of the second arm 42 has a distal tip 60 opposing the distal tip 56 of the first arm 40. The distal tips 56, 60 are fabricated from a magnetic material such that the distal tips 56, 60 are magnetically attracted to one another. For example, the distal tip 56 of the first arm 40 may be fabricated from a permanent magnet or a metal and the distal tip 60 of the second arm 42 may be fabricated from the other of a permanent magnet or a metal. In aspects, each of the distal tips 56, 60 are permanent magnets. The magnetic attraction between the distal tips 56, 60 facilitates a locking closure of the distal end portions 40b, 42b of the arms 40, 42 about tissue and completes a circuit within the arms 40, 42, as will be described in further detail herein.

The intermediate segment 58 of the distal end portion 40b of the first arm 40 may be flat at the tissue-contacting surface 46 to allow for maximum tissue contact and has a plurality of sensors 62 housed therein or supported thereon. The distal end portion 42b of the second arm 42 has a plurality of emitters 64 housed therein or supported thereon. The sensors 64 may be a single or a plurality of photodetectors, and the emitter 64 may be a single or a plurality of infrared or LED emitters configured to emit a signal sensed by the photodetectors 64. Other suitable types of optical sensing sensors are also contemplated including broad band light sources, and laser diodes (LDs), and light receivers including photodiodes (PDs) and silicon photomultipliers (SiPMs), CCD arrays, CMOS imaging sensors, cameras, and spectrometers. The sensors 62, based on the signals emitted by the emitters 64 which pass through grasped tissue, are configured to measure at least one of oxygen concentration, oxygen pressure, tissue perfusion, tissue flow dynamics, tissue chemical composition, tissue immunologic activity, tissue pathogen concentration, or tissue water content.

The sensors 62 and emitters 64 are in communication, via lead wires 68, 70 or wireless connection, with the a computing device or processor “P” in the handle portion 12, which processes the information collected by the sensors 62 to calculate or determine the tissue property being measured. The processor “P” may also be in communication, via lead wires or wireless connection, with a display (not shown) in the handle portion 12 to display the determined tissue properties. The lead wires 68, 70 may be a flexible circuit that extends distally from the processor “P” to the respective sensors 62 and emitters 64. The flexible circuits 68, 70 may form a single, closed circuit upon the magnetic distal tips 56, 60 engaging one another to allow for the transference of data between the sensors 62 and emitters 64 and/or from the sensors 62 and emitters 64 to the processor “P.” The processor “P” may be configured to detect when the magnetic distal tips 56, 60 are engaged to one another and in response, configured to activate the emitters 64 to send the signals (e.g., LED light or infrared) toward the sensors 62. A battery (not shown) may be provided in the handle portion 12 to provide power to the sensors 62 and emitters 64 via the wires 68, 70 when the circuit is closed.

In operation, the tissue grasper 10 may be used prior to, during, or after a surgical procedure, for example, an anastomotic surgical procedure, to gather various data about the subject tissue. In an anastomotic surgical procedure, for example, colorectal surgery, unhealthy or diseased bowel tissue is resected and the ends of the remaining healthy segments of bowel are stapled together to recreate a continuous bowel. Prior to stapling the ends of the separate bowel segments to one another, the viability of the ends of the separate bowel segments should be assessed in order to predict the likelihood of post-surgery anastomotic leaks or other adverse outcomes. To aid in making this viability assessment, a surgeon may make use of the tissue grasper 10 of the present disclosure.

In use of the tissue grasper 10, the tissue grasper 10, with the distal end portions 40b, 42b of the arms 40, 42 received in the shaft 30 and thereby assuming the collapsed state, may be passed through a port into a surgical site. Upon entering the surgical site, the distal end portions 40b, 42b may be advanced out of the shaft 30 to allow the distal end portions 40b, 42b to radially expand to their unbiased arcuate shapes. As shown in FIG. 2, each of the two ends of the presumably healthy bowel segments “B1,” “B2” are grasped, either separately or together, between the tissue contacting surfaces 46, 48 of the arms 40, 42. The distal tips 56, 60, due to the magnetic attraction therebetween, approximate toward one another and ultimately engage one another to close about the tissue. In aspects, the arms 40, 42 may be partially retracted within the shaft 30 to facilitate approximation of the distal tips 56, 60. Upon the distal tips 56, 60 engaging one another, the processor “P” initiates a tissue sensing protocol, whereby the emitters 64 emit signals through the tissue and the sensors 62 receive the signals. The received signals are sent from the sensors 62 to the processor “P” to determine a tissue property of the grasped tissue, such as the oxygen concentration or the tissue perfusion. A clinician may then use this information to determine whether the tissue grasped is viable or whether more tissue needs to be resected.

The tissue grasper 10 may also be configured to be incorporated into a robotic surgical system (not shown). The robotic surgical system is powered locally or remotely, and has electronic control systems localized in a console or distributed within or throughout the robotic surgical system. The robotic surgical system permits a surgeon to remotely manipulate the tissue grasper 10 to more precisely control the movement of the tissue grasper 10. The tissue grasper 10 may be configured to send the measurements gathered by the sensors to an interface of the robotic surgical system on which the measurements may be displayed for the surgeon to read.

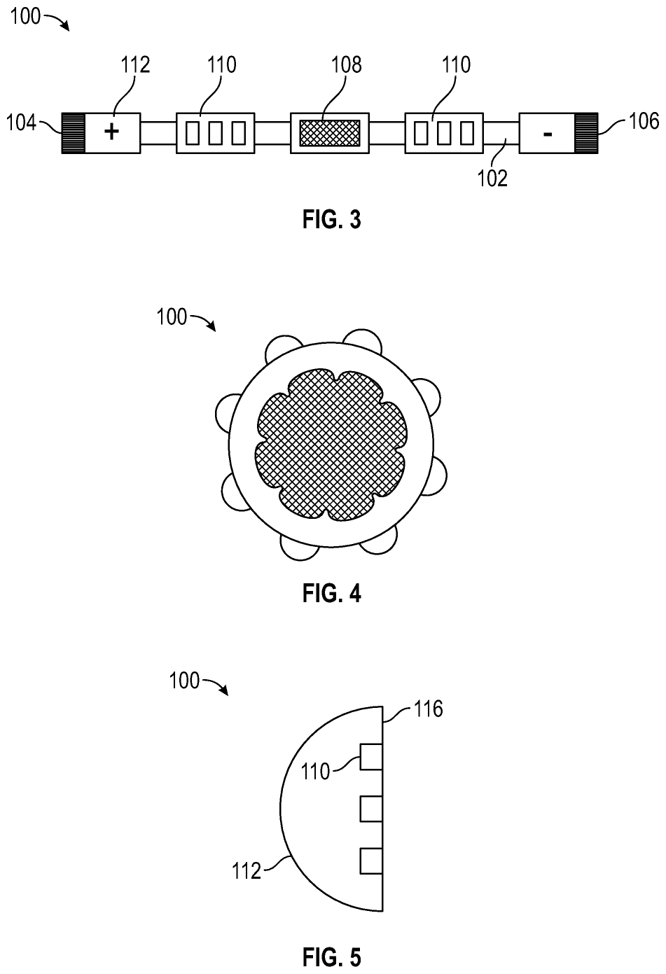

With reference to FIGS. 3-7, another device for measuring tissue properties during an anastomotic surgical procedure or other suitable procedure is illustrated. The device is a bracelet or collar 100 equipped with an array of sensors, such as, for example, IR or near-IR photodiodes and detectors configured to interrogate tissue or organs of interest. The bracelet 100 may be flexible to be used on varying sizes and challenging tissues or organs that require circumferential evaluation. The bracelet 100 allows for monitoring tissues/organs that are hidden from the visual field for signs of early necrosis or reduced perfusion. The bracelet 100 may comprise a plurality of discrete units detachably coupled to one another so that the circumference of the bracelet 100 may be selectively adjusted by adding or removing the discrete units. The bracelet 100 may be introduced via a laparoscopic port and assembled using any suitable laparoscopic tool or grasper in open procedures installed using the clinician's hands or a robotic instrument.

The bracelet 100 includes a flexible cable or circuit 102 and first and second ends 104, 106 that are detachably coupled to one another via a magnetic connection, similar to the distal tips 56, 60 of the surgical instrument 10 of FIGS. 1-2. The bracelet 100 may be transitioned between an opened state (FIG. 3), in which the ends 104, 106 of the bracelet 100 are decoupled from one another, and a closed state (FIG. 4), in which the ends 104, 106 are coupled to one another and the bracelet 100 assumes a closed loop configuration.

The bracelet 100 includes a plurality of sensors 108 (e.g., photodetectors) disposed in a linear array on a first half of the bracelet 100, and a plurality of emitters 110 (e.g., LED or infrared emitters) disposed in a linear array on a second half of the bracelet 100. As such, upon closing the bracelet 100 about tissue, the sensors 108 and emitters 110 oppose one another to allow for signals emitted from the emitters 110 to travel through the tissue and toward the sensors 108. The bracelet 100 may include a battery 112 for powering the sensors 108 and emitters 110. The bracelet 100, when in a closed state, has an arcuate outer surface 114, and a flat inner surface 116 to maximize the tissue contact and to maintain the rotational orientation of the bracelet 100 relative to the tissue.

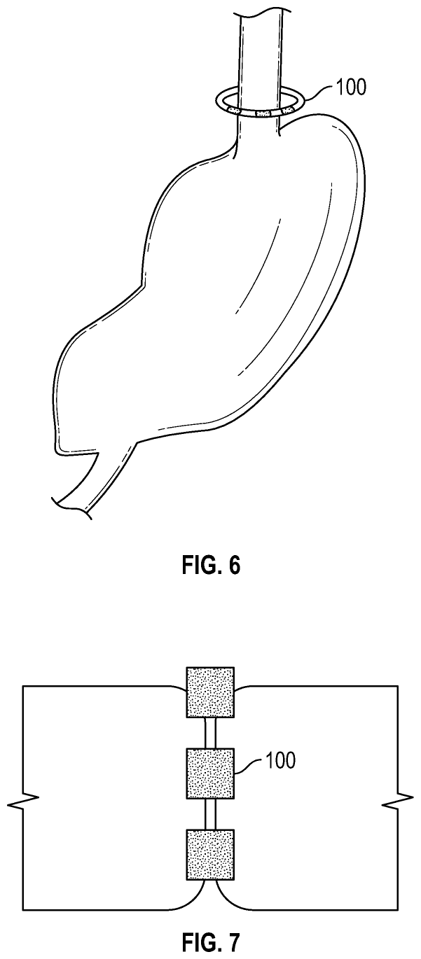

In operation, the bracelet 100, while in the opened state (FIG. 3), may be positioned about tissue, such as, for example, esophageal tissue (FIG. 6) or an anastomosis (FIG. 7), and the ends 104, 106 of the bracelet 100 are coupled to one another to close the flexible circuit 102. Upon closing the circuit, the battery 112 provides power to the emitters 110 to emit the signals through the tissue to the sensors 108. The sensors 108 may be in communication with a memory (not explicitly shown) of the bracelet 100 or may be in wireless communication with a computer in the surgical suite that receives the measurements from the sensors 108 to determine the tissue properties.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical instrument, comprising:
an elongated shaft defining a passageway therethrough; and
a pair of first and second elongated arms extending through the shaft, each of the first and second arms having a generally arcuate, distal end portion configured to extend distally out of the shaft and to surround tissue, the distal end portion of the first arm having a sensor configured to sense a property of the tissue, wherein each of the first and second arms has a distal tip fabricated from a magnetic material such that the distal tips are magnetically attracted to one another.

2. The surgical instrument according to claim 1, wherein the distal end portion of the second arm has an emitter configured to emit a signal sensed by the sensor.

3. The surgical instrument according to claim 2, wherein the sensor is a photodetector, and the emitter is an infrared or LED emitter.

4. The surgical instrument according to claim 2, further comprising a processor in communication with the sensor and the emitter, wherein the processor is configured to determine the property of the tissue based on information received from the sensor.

5. The surgical instrument according to claim 4, wherein the processor is configured to cause the emitter to emit the signal upon the distal tips engaging one another.

6. The surgical instrument according to claim 1, wherein the distal tip of the first arm and the distal tip of the second arm are oriented toward one another.

7. The surgical instrument according to claim 1, wherein the first and second arms are configured to translate relative to the shaft between a retracted position, in which the distal end portion of each of the first and second arms is received within the shaft, and an extended position, in which the distal end portion of each of the first and second arms extends distally out of the shaft.

8. The surgical instrument according to claim 1, wherein the distal end portion of the first arm has a linear segment fabricated from a rigid material, and a remainder of the distal end portion of the first arm is arcuate and fabricated from a flexible material.

9. The surgical instrument according to claim 8, wherein the linear segment houses the sensor.

10. The surgical instrument according to claim 8, wherein the distal end portion of the second arm is fabricated from the flexible material.

11. The surgical instrument according to claim 10, wherein the flexible material is a shape memory material such that the distal end portion of the first and second arms are biased toward a generally arcuate shape.

12. The surgical instrument according to claim 1, wherein the tissue property is at least one of blood perfusion, oxygen concentration, or oxygen pressure.

13. A tissue grasper for sensing tissue properties, the tissue grasper comprising:
a handle portion having a processor;
an elongated shaft extending distally from the handle portion; and
a pair of first and second elongated arms extending through the shaft, each of the first and second arms having a distal end portion configured to extend distally out of the shaft and to surround tissue, the distal end portion of the first arm having a sensor in communication with the processor and configured to sense a property of the tissue, the distal end portion of the second arm having an emitter configured to emit a signal sensed by the sensor.

14. The tissue grasper according to claim 13, wherein the first arm has a magnetic distal tip, and the second arm has a magnetic distal tip such that the distal tips are magnetically attracted to one another.

15. The tissue grasper according to claim 14, wherein the distal tips are arcuate and oriented toward one another.

16. The tissue grasper according to claim 13, wherein the sensor is a plurality of photodetectors, and the emitter is a plurality of infrared or LED emitters.

17. The tissue grasper according to claim 16, wherein the processor is in communication with the plurality of emitters and configured to determine the property of the tissue based on information received from the plurality of photodetectors.

18. The tissue grasper according to claim 17, wherein the processor is configured to cause the plurality of emitters to emit the signal upon the distal end portions of the first and second arms engaging one another.

19. The tissue grasper according to claim 13, wherein the first and second arms are configured to translate relative to the shaft between a retracted position, in which the distal end portion of each of the first and second arms is received within the shaft, and an extended position, in which the distal end portion of each of the first and second arms extends distally out of the shaft.

20. The tissue grasper according to claim 13, wherein the distal end portion of the first arm has a linear segment fabricated from a rigid material, and a remainder of the distal end portion of the first arm is arcuate and fabricated from a flexible material.
